SEQUENCE LISTING (2 sequences)

SEQ ID NO: 1 - DNA, 1121 bp, JCV
  CDS (39)..(1100)
gtacgggactgcagcacctgctcttgaagcatatgaagatggccccaacaaaaagaaaaggagaaaggaaggaccccgtgcaagttccaaaacttcttataagaggaggagtagaagttctagaagttaaaactggggttgactcaattacagaggtagaatgctttttaactccagaaatgggtgacccagatgagcatcttaggggttttagtaagtcaatatctatatcagatacatttgaaagtgactccccaaatagggacatgcttccttgttacagtgtggccagaattccactacccaatctaaatgaggatctaacctgtggaaatatactcatgtgggaggctgtgaccttaaaaactgaggttataggggtgacaagtttgatgaatgtgcactctaatgggcaagcaactcatgacaatggtgcagggaagccagtgcagggcaccagctttcattttttttctgttgggggggaggctttagaattacagggggtgctttttaattacagaacaaagtacccagatggaacaatttttccaaagaatgccacagtgcaatctcaagtcatgaacacagagcacaaggcgtacctagataagaacaaagcatatcctgttgaatgttgggttcctgatcccaccagaaatgaaaacacaagatattttgggacactaacaggaggagaaaatgttcctccagttcttcatataacaaacactgccacaacagtgttgcttgatgaatttggtgttgggccactttgcaaaggtgacaacttatacttgtcagctgttgatgtctgtggcatgtttacaaacaggtctggttcccagcagtggagaggactctccagatattttaaggtgcagctaaggaaaaggagggttaaaaacccctacccaatttctttccttcttactgatttaattaacagaaggactcctagagttgatgggcagcctatgtatggcatggatgctcaagtagaggaggttagagtttttgagggaacagaggagcttccaggggacccagacatgatgagatacgttgacaaatatggacagttgcagacaaaaatgctgtaatcaaaagcttttattgta

SEQ ID NO: 2 - protein, 354 aa, JCV
MAPTKRKGERKDPVQVPKLLIRGGVEVLEVKTGVDSITEVECFLTPEMGDPDEHLRGFSKSISISDTFESDSPNRDMLPCYSVARIPLPNLNEDLTCGNILMWEAVTLKTEVIGVTSLMNVHSNGQATHDNGAGKPVQGTSFHFFSVGGEALELQGVLFNYRTKYPDGTIFPKNATVQSQVMNTEHKAYLDKNKAYPVECWVPDPTRNENTRYFGTLTGGENVPPVLHITNTATTVLLDEFGVGPLCKGDNLYLSAVDVCGMFTNRSGSQQWRGLSRYFKVQLRKRRVKNPYPISFLLTDLINRRTPRVDGQPMYGMDAQVEEVRVFEGTEELPGDPDMMRYVDKYGQLQTKML

(19) United States
(12) Patent Application Publication
(10) Pub. No.: US 2003/0044961 A1
(43) Pub. Date: Mar. 6, 2003

(54) Compositions for transferring active compounds in a cell-specific manner

(76) Inventors: Wolfgang Luke, Gottingen (DE); Harald Petry, Gleichen (DE); Oliver Ast, Dransfeld (DE); Ingo Wilke, Friedland Ballenhausen (DE); Claudia Goldmann, Gottingen (DE); Kerstin Wagner, Jena (DE); Matthias Schnabelrauch, Jena (DE)

Correspondence Address: STRIKER, STRIKER & STENBY, 103 East Neck Road, Huntington, NY, 11743, US

(21) Appl. No.: 10/179,126

(22) Filed: Jun. 26, 2002

(30) Foreign Application Priority Data
Jun. 28, 2001 (DE) 101 31 145.1

(51) Int. Cl. C12N 15/86; C12N 7/00

(52) U.S. Cl. 435/235.1; 435/456; 435/320.1

(57) ABSTRACT

The composition for the cell-specific transfer of an active compound in specific target cells can be used as a diagnostic or therapeutic agent or for gene therapy. The composition includes virus-like particles, which are each composed of a number of viral protein molecules derived from JC virus, a cationic polymer, for example a polyamine, a polyimine or an amino acid polymer, especially polyethylenimine, as an anchor molecule for a cell-specific ligand, and preferably a ligand bound to the cationic polymer as a binding partner for a cell-specific receptor. The viral protein is advantageously encoded with a nucleic acid having nucleotide sequence as shown by SEQ. ID NO. 1.

### BACKGROUND OF THE INVENTION

1. Field of the Invention

The invention relates to novel compositions for the cell-specific transfer of active compounds, which compositions are based on viral structural proteins that are associated with a cationic polymer as an anchor for binding ligands, in particular target cell-specific ligands.

2. Description of the Related Art

The clinical practicability of recombinant methods essentially depends on the efficiency, target cell specificity and biological safety of the transfer system that is used for introducing the therapeutic DNA into the cells. All the transfer systems that have thus far been made available suffer from significant problems with regard to these properties. Because of the potential danger of recombination with cellular sequences, viral transfer systems involve a safety risk that is hardly possible to calculate. On account of their high degree of immunogenicity in most patients, adenoviruses and adeno-associated viruses, which are currently the favored systems for transporting therapeutic genes, render repeated in vivo administration impossible. While non-viral systems, such as liposomes and DNA-condensing molecules, avoid these disadvantages, their transfer efficiencies and target cell specificities are, on the other hand, like those of retroviral systems, far lower.

A novel transfer system based on virus-like particles (VLPs) has been developed to circumvent these problems (WO 97/19174). These VLPs can be prepared by recombinantly expressing the main structural protein VP1 of the human polyomavirus JCV in insect cells. This system is based on the property possessed by VP1-VLPs of being able to package DNA and then inserting this DNA specifically into particular cells. In contrast to the expression of VP1 from other polyomaviruses, VP1-VLPs are secreted into the cell culture supernatant, from which they are prepared in high purity by means of two consecutive centrifugation steps. VP1-VLPs can be dissociated into VP1 pentamers by removing Ca²⁺ ions under reducing conditions and, in contrast to the VP1-VLPs from other polyomaviruses, can be subsequently re-associated once again into complete VP1-VLPs. The DNA is packaged during this VP1 re-association process, under defined in vitro conditions, without the morphological and biological properties of the VP1-VLPs being altered during this process. The DNA, which has been packaged in this way, is subsequently protected from being enzymatically degraded by DNase I. In addition to this, it has been shown that, apart from the DNA, low molecular weight substances can also be packaged into the VP1-VLPs. Using the VP1-VLPs, the packaged foreign DNA is efficiently and specifically inserted into cells of renal and neuronal origin and expressed in these cells. This cell tropism of the VP1-VLPs, which is very narrow in contrast to other transfer systems, corresponds to that of natural JC virus and is a very advantageous feature when using the VP1-VLPs as a DNA transfer system.

In addition to biological safety and transfer efficiency, the target cell specificity of transfer systems is one of the crucial criteria for using these systems in vivo for treating diseases by means of gene therapy. Many of the currently available viral and non-viral transfer systems have a very broad host cell spectrum. Attempts to restrict the tropism of the systems to particular cells or tissues are made by selectively altering or substituting coat proteins. On the one hand, these structural alterations are very elaborate to perform and, on the other hand, are frequently achieved at the expense of transfer efficiency.

The suitability of the VP1-VLPs for use as a cell-specific system for transporting and transducing DNA has been investigated in a variety of cell lines. Immunofluorescence investigations have shown that the VP1-VLPs bind exclusively to cells of renal and neuronal origin, and are internalized, and transported into the cell nucleus, within a short period of time.

### SUMMARY OF THE INVENTION

The invention consequently relates to the use of VP1-VLPs for specifically transducing cells of renal and neuronal origin.

The invention, which is described here additionally, relates to a method for selectively altering the cell tropism of the VP1-VLPs in order, in this way, to selectively transduce very specific target cells and tissues. This principle makes it possible to use the VP1-VLPs flexibly as a DNA transfer system when treating diseases, which are restricted to particular cell types or tissues.

Against this background, it has been found, surprisingly, that the target cell specificity can be altered selectively by loading the VP1-VLPs with cationic polymers as anchor molecules for cell-specific ligands. It is therefore possible to use the VP1-VLPs as a cell-specific transport system for therapeutic nucleic acids or substances. In the light of the fact that the VP1 protein is produced recombinantly, and separately from the therapeutic DNA, in large quantities and at high purity, and that the DNA packaging does not take place in packaging cell lines, as it does in the case of retroviral, adeno-associated or adenoviral vectors, it is possible to exclude contamination with viral nucleic acids and the potential danger of infectious viruses being formed as a result of recombination events. Since, furthermore, the DNA packaging, and the loading of the VP1-VLPs with cell-specific ligands take place under defined in vitro conditioned, the VP1-VLP DNA transfer system constitutes a biologically safe platform technology which combines the advantages of viral and non-viral systems without, however, suffering from their disadvantages.

The invention consequently relates to conjugates of virus-like particles (VLPs), which are composed of several molecules of the JC virus viral protein VP1, which are associated with a cationic polymer which can serve as an anchor for binding other ligands. The VLPs according to the invention are distinguished, in particular, by the fact that they are free of JCV-associated nucleic acids. VLPs of this nature, in particular VLPs composed of recombinant VP1 molecules, are described in WO 97/19174.

JVC VP1 is the main structural protein in the capsid coat of JCV, e.g. as obtained from wild-type strains or mutagenized strains of JCV. In a particular embodiment, the VLP is composed of recombinantly prepared VP1. The term VP1 therefore also encompasses proteins, which differ from wild-type VP1 as the result of mutations, such as substitutions, insertions and/or deletions.

In order to prepare recombinant VP1, use is preferably made of a nucleic acid, which comprises the sequence shown in SEQ. ID NO. 1, or a sequence, which is complementary to this, a sequence which corresponds to this sequence within the context of the degeneracy of the genetic code, or a sequence which hybridizes with it under stringent conditions, with the nucleic acid sequence, or a recombinant vector which contains this sequence, being introduced into a suitable host cell, the host cell being cultured under conditions under which the nucleic acid sequence is expressed, and the protein being isolated from the cell or the cell supernatant. Stringent hybridization conditions are preferably defined as described by Sambrook, et al, (1989) Molecular Cloning, A Laboratory Manual, Second Edition, Cold Spring Harbor Laboratory Press, and include a washing step of 30 mm in 0.1×SSC, 0.5% SDS at 60° C. and preferably 68° C.

In addition, the VLP according to the invention can have one or more additional heterologous proteins in the capsid structure. This is to be understood as meaning that a heterologous protein is anchored in the capsid structure, with preferably at least a part of this protein being accessible from the outside. In principle, all proteins which can be incorporated into the capsid structure, and which do not impair the self-assembly of the VLP are heterologous proteins which are suitable for this purpose.

The cationic polymer, which is associated with the VLP, is preferably a physiologically compatible polymer. Examples of suitable cationic polymers are polyamines and/or polyimines, i.e. polymers, which contain primary, secondary or tertiary amino and/or imino functional groups in adequate quantity for ensuring that the polymer has a positive net charge under physiological conditions. Advantageously, the ratio of cationic groups to anionic groups is greater than or equal to 2:1, particularly preferably greater than or equal to 5:1. Most preferably, the cationic polymer is essentially free of anionic groups. The molecular weight of the cationic polymers is preferably in the range of from 10 to 750 kD, particularly preferably in the range of from 25 to 100 kD.

Specific examples of cationic polymers are polymers, which are essentially based on basic amino acids, such as polylysine, in particular poly-L-lysine, etc. Other specific examples of suitable cationic polymers are polyalkylenimine, preferably poly-C₂-C₄-alkylenimines, in particular polyethyleneimine (PEI), pAMAM (polyamido-amine) dendrimers and fractionated dendrimers, and also cationically modified polyethylene glycol. Polyethyleneimine is a particularly preferred cationic polymer within the meaning of the present invention, since it is not toxic and has a high density of positive charges. PEI is furthermore able, after having been taken up into the cells, to bring about a pH-dependent structural change, which leads to the destabilization of endosomal and lysosomal cell compartments and consequently facilitates the release of active compounds, e.g. nucleic acids, into the cytoplasm. This process is supported by the pronounced buffering capacity of the imino groups, which are protonated after acidification in the lysosomes and then give rise to osmotic rupture of the vesicle membrane.

It has now been found, within the context of the present investigations, that polycations have a high affinity for VP1-VLPs. The preferred ratio by weight of VP1-VLP to cationic polymer in the conjugates according to the invention can be varied within wide ranges. Thus, ratios by weight of from 5:1 to 1:10 have proved to be suitable, with ratios by weight of 2:1 and 1:5 being particularly preferred for making it possible to achieve optimum binding.

In a preferred embodiment of the invention, at least one ligand is bound to the cationic polymer, which is associated with the VP1-VLP. In principle, the ligand may be any arbitrary substance, provided it can be bound directly or indirectly to the cationic polymer by way of covalent and/or noncovalent interactions. For example, the ligand can be a target cell-specific group, e.g. a binding partner for a cell surface receptor. Suitable examples of binding partners are natural ligands or synthetic analogues of these ligands, with it being possible to use high molecular weight ligands, such as proteins, e.g. transferrin, antibodies or sugars, such as mannose, or else low molecular weight synthetic ligands, e.g. the tripeptide motif R-G-D (Arg-Gly-Asp). Alternatively or in addition, it is also possible to use a labeling group, e.g. a group that can be recognized by suitable detection methods, such as a fluorescence labeling group or biotin, as the ligand. Furthermore, the ligand can also be an effector group, e.g. a cytotoxic group. It is naturally also possible to use combinations of several ligands, in particular combinations of the previously mentioned ligands.

In a special embodiment, the VLP can contain one or more active substances within the capsid structure. In this description, “active substance” is understood as meaning any molecule which is not customarily present in the medium which is used in connection with the self-assembly. These active substances include, for example, macromolecules, such as nucleic acids, i.e. RNA, DNA or artificial, modified nucleic acids, and also proteins and other physiologically active substances, which can be of a natural, synthetic or recombinant nature. Examples of such physiologically active substances are lipids, phospholipids, peptides, drugs, toxins, etc.

In another aspect, the invention relates to a process for preparing conjugates composed of VP1-VLP and cationic polymers, with several VP1 molecules being assembled into a particle and a cationic polymer being added, for association with the particle, during and/or after the assembly.

The cationic polymer is preferably added after the assembly. Particularly preferably, the VLPs, in particular the recombinant VLPs, are first of all purified, then dissociated and subsequently re-associated in the presence of the active substance. If the conjugate is to contain additional active substances as well, the assembly is then preferably carried out in the presence of this additional substance, which is then enclosed in the interior of the VLP capsid coat.

Preference is given to the VLP being prepared recombinantly with a VP1-protein-coding nucleic acid being introduced into a cell, the transformed cell being cultured in a medium under conditions under which the nucleic acid is expressed, and the expression product being isolated from the cell or from the medium. The recombinant VP1 is isolated directly from the host cells and/or from the cell culture supernatant depending on the host-vector system employed. The particular advantage of the recombinant method is that it is readily possible to obtain VLPs in high purity and in large quantities. The use of baculoviruses in combination with insect cells, e.g. with the insect cell line Sf 158, has in practice proved to be an expression system of choice.

In order to prepare VLPs which have incorporated a heterologous protein within the capsid structure, or VLPs, which contain an active substance within the capsid structure, the above preparation process is modified in that the heterologous proteins and/or active substances are added, in the desired quantity or concentration, at a suitable time point, i.e. before the VLPs are assembled, and the assembly is then allowed to take place. In this way, it is possible to form VLPs, which have incorporated heterologous protein in the capsid coat and/or contain an enclosed active substance, e.g. a nucleic acid, in the interior. Heterologous polypeptides can, for example, be incorporated in the capsid coat by recombinantly co-expressing the respective polypeptides, i.e. the VP1 polypeptide and the heterologous polypeptide, in a suitable host cell, for example a eukaryotic cell. Active substances can, for example, be incorporated into the interior of the capsid coat by dissociating the capsid coat and subsequently re-associating it in the presence of the active substance, or by subjecting the VLPs to osmotic shock in the presence of the active substance.

The conjugates according to the invention, composed of VLPs and cationic polymer, can be used for diagnostic and therapeutic purposes, for example for diagnosing and treating diseases, such as PML, which are associated with an infection with JC virus.

In another embodiment, the VLPs can be used as a transport vehicle, in particular for transporting active compounds to a target cell and, preferably into the target cell. In this connection, the target cell specificity is significantly altered, as compared with that of unmodified VLP, by binding a ligand to the cationic polymer, which is associated with the VLP.

In this way, it is possible to ensure the specificity of the interaction with the designated target cells and, in dependence on the application, match this specificity to a large number of cell types. An example of such a use is the selective transport of TNF-α antisense nucleic acids to oligodendrocytes in multiple sclerosis, since it is known that, in this disease, expression of TNF-α during an episode leads to demyelination. Another example is the use of VLPs as a system for transporting nucleic acids in gene therapy.

Inserting the herpes virus thymidine kinase gene initiates the suicide mechanism of the cell. Selectively transporting the Tk (thymidine kinase) gene into neoplastically transformed cells, such as benign prostate hyperplasia cells, results, after a nucleoside analog (e.g. acyclovir or gancyclovir) has also been added, in replication being terminated and, following on from this, in the transduced cells dying.

The invention is now further clarified by means of the following examples and the enclosed drawing and sequence listings.

### BRIEF DESCRIPTION OF THE SEVERAL VIEWS OF THE DRAWING

The objects, features and advantages of the invention will now be illustrated in more detail with the aid of the following description of the preferred embodiments, with reference to the accompanying figures in which:

FIGS. 1A and 1B show VP1-specific immunofluorescence for detecting VP1-VLP obtained by incubation 5×10⁴ of SVG and COS-7 cells for 24 hours, fixing them and detecting VP1 by fluorescence microscopic methods, using a VP1-specific immune serum and subsequently incubating with an FITC-coupled anti-rabbit Mab;

FIG. 2 is a graphical illustration of luciferase activity in cell lysates of different types of cells determined luminometrically;

FIGS. 3 and 4 are respective graphical illustrations of the optical absorptivity as a function of PEI-biotin concentration measured photometrically at 490 nm in corresponding determinations of the ability of PEI to bind to VP1-VLP in ELISA tests;

FIG. 5 shows VP1-specific immunofluoescence for detecting VP1-VLP and VP1-VLP/PEI-transferrin complexes in HeLa, DU-145 and EM 1604 cells; and

FIG. 6 is a graphical illustration of luciferase activity in cell lysates of different types of cells determined luminometrically for the purpose of measuring transduction efficiency of VP1-VLP/PEI-transferrin complexes in various cell lines.

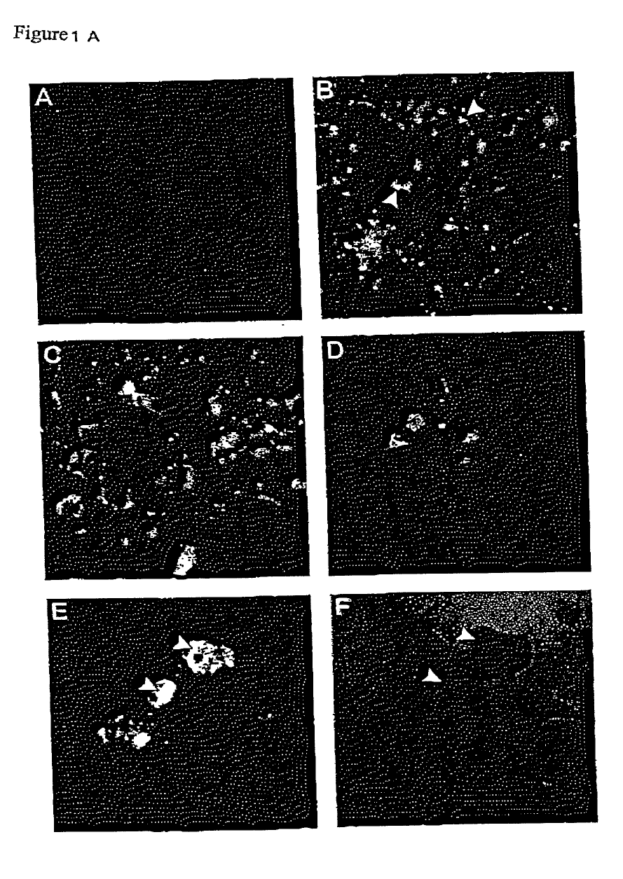

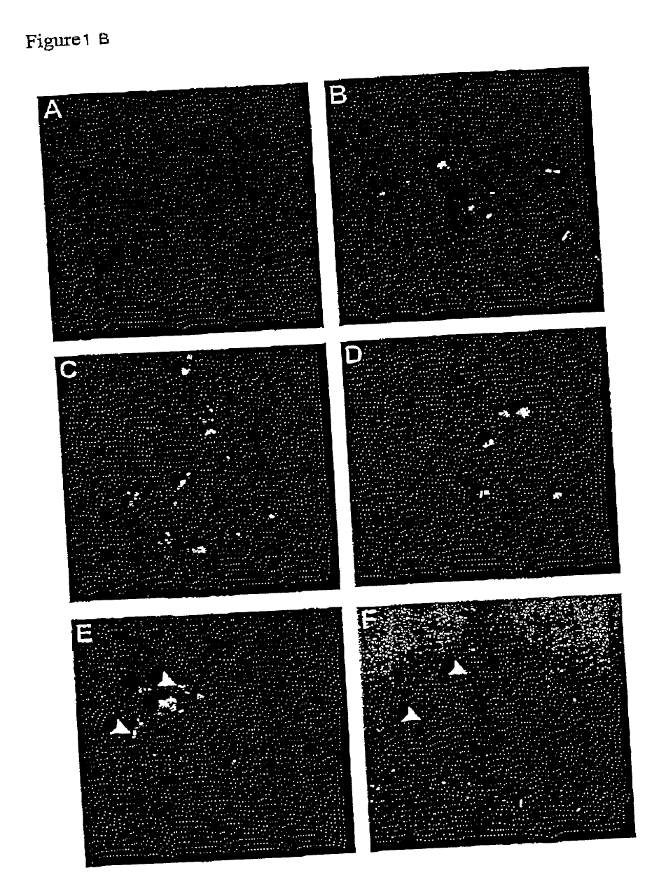

### EXAMPLES

### Example 1

### Specifically Transducing Cells of Neuronal and Renal Origin with JCV-VLP

It was observed that the JCV-VLPs have a specific tropism for cells of renal and neuronal origin. For the transduction experiments, the JCV-VLPs were purified from the supernatant of an insect cell line (SF158), which had been infected with VP1-recombinant baculoviruses. For packaging the DNA, 50 μg of purified VP1-VLPs were dissociated in a total volume of 100 μl containing 10 mM Tris-HCl, pH 7.5, 10 mM EGTA, 150 mM NaCl and 5 mM DTT. In order to package the DNA, VP1 pentamers were dialysed, in the presence of the plasmids to be packaged, against a Ca²⁺-containing buffer in order to dilute the sequestering agent EGTA and the reducing agent DTT and to supply Ca²⁺ ions at the same time. Electron microscopy was used to detect the formation of the JCV-VLPs. A DNase I digestion was carried out in order to remove the plasmid DNA, which was bound to the surface of the VP1-VLPs. For each reaction assay, 10 U of DNase I (Pharmacia Biotech, Freiburg) were added, final concentrations of 10 mM Tris-HCl, pH 7.5, and 6 mM MgCl₂, were established, and the mixture was incubated at 37° C. for one hour.

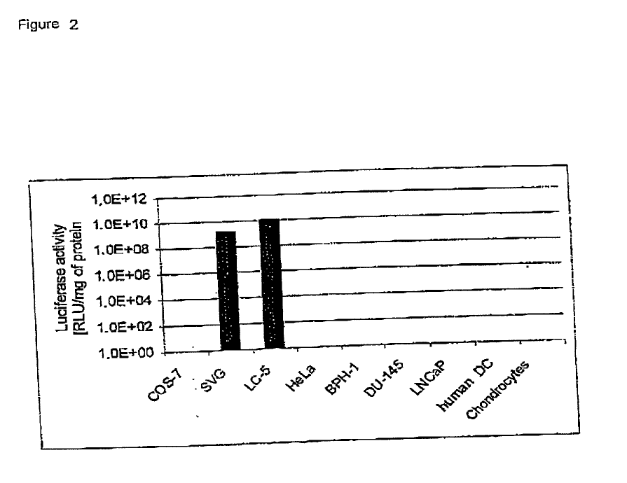

It was possible to use these JCV-VLPs to specifically transduce cells of neuronal origin (such as SVG cells) and renal origin (such as COS-7 cells). For this, in each case 5×10⁴ cells were incubated overnight for 24 hours, at 37° C., with the DNA-containing VP1-VLPs. The reporter gene luciferase was used to determine the transduction efficiency by means of luminometry. In order to determine the transduction efficiency of VP1-VLP, 1 μg of the DNA of the reporter plasmid pGL3-C was packaged in 1.25 μg of VP1-VLP. In these experiments, high transduction efficiencies were measured in cells of neuronal (SVG cells) and renal origin (COS-7 cells), as shown in FIG. 2. On the other hand, no luciferase activities were observed in fibroblasts, T lymphocytes, dendritic cells, chondrocytes, cells from the prostate or from mammary carcinomas, which had likewise been incubated with the JCV-VLPs.

### Example 2

### Characterizing the PEI-VLP Binding Properties

Since the degree of polymerization of the PEI and the coupling density of the ligands have consequences for the charge distribution in the molecule, the properties of two different PEI preparations (25 kD polymer from SlGMA/Aldrich, Deisenhofen, Germany) with regard to their binding to the VP1-VLPs were investigated in an ELISA test.

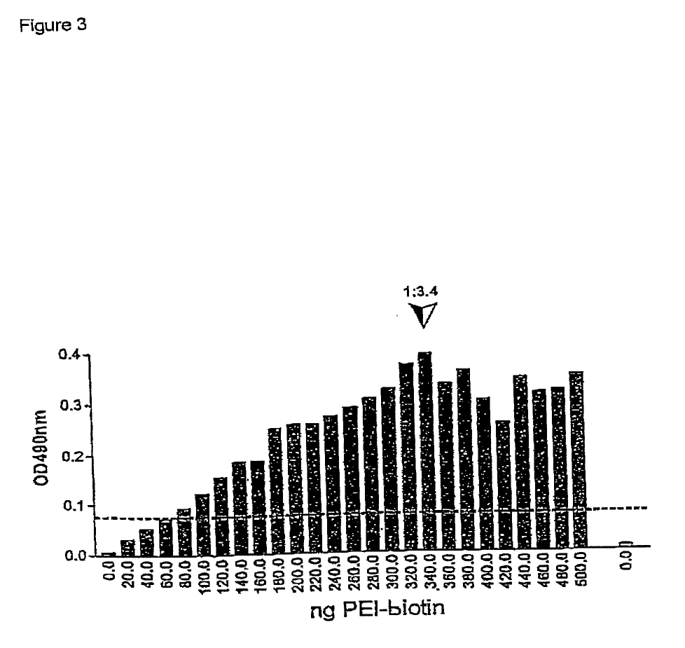

For this, biotin was first of all coupled covalently, as a readily detectable group, to polyethylenimine in a molar ratio of 1:1. The ability of the PEI-biotin to bind to the VP1-VLPs was determined by binding 100 μg of VP1-VLP or 100 μg of VP1 per well in a 96-well ELISA plate. This VP1-VLP was incubated with PEI-biotin at various concentrations. Then a horseradish peroxidase (HPR)-conjugated streptavidin conjugate was used to quantify the binding photometrically at 490 nm. This gave an optimum VP1-VLP/PEI-biotin binding ratio (w/w) of 1:3.4, as shown in FIG. 3. The broken line in FIG. 3 marks the cut-off value of the measurement.

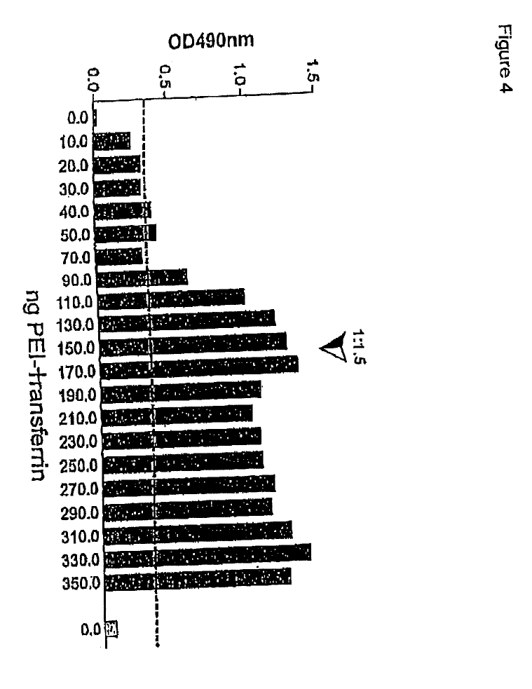

Analogous experiments were carried out using PEI-transferrin (Q-Biogene, Heidelberg, Germany). For this, 100 ng of VP1-VLPs or 100 μg of VP1 were once again bound per well on an ELISA plate and incubated with various concentrations of PEI-transferrin; a monoclonal anti-transferrin antibody was then used to detect the binding quantitatively at 490 nm. This gave an optimum VP1-VLP/PEI-transferrin binding ratio (w/w) of 1:1.5, as shown in FIG. 4. The broken line in FIG. 4 marks the cut-off value of the measurement.

### Example 3

### Cell Binding Test Using JCV VP1-VLP/PEI-Transferrin

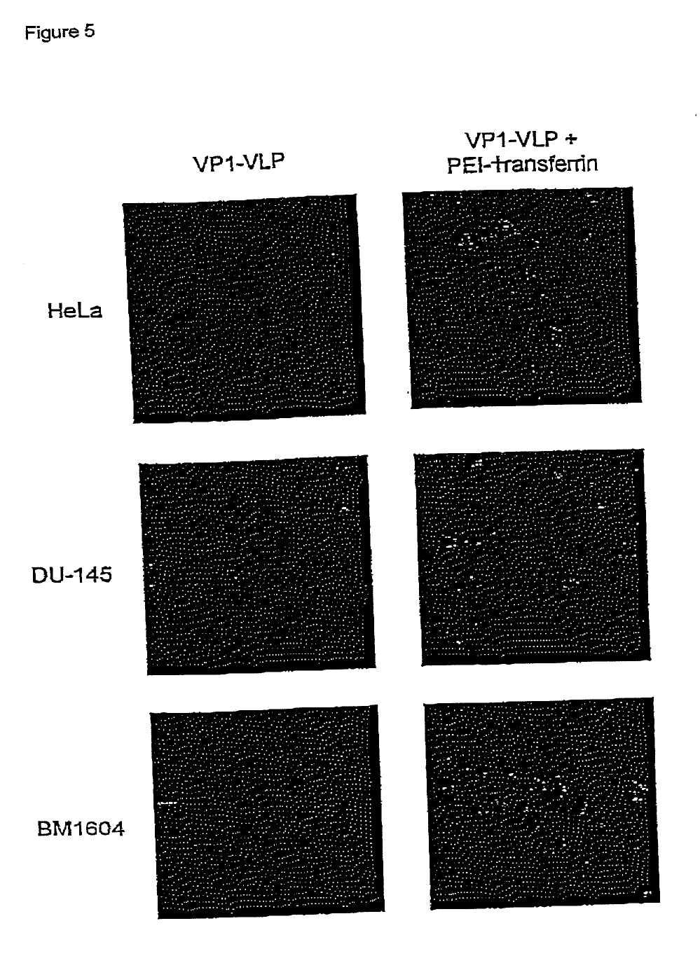

The binding, the internalization and the nuclear transport of the VP1-VLP/PEI-transferrin complexes were established in cell binding tests. The VP1-VLPs were loaded in a simple in vitro system. The VP1-VLP/PEI transferrin weight ratio used for this was 1:1.5. The binding took place during a 30-minute incubation at 37° C. The cell binding tests were carried out on the cell lines HeLa (Scherer et al, J. Exp. Medicine 97, (1953), 695), DU145, BM 1604 and BPH-1 (Mitchell et al., BJU International 85 (2000), 932), which express the transferrin receptor. For this, in each case 5×10⁴ cells were incubated with 3 μg of VP1-VLP/PEI transferrin for 24 hours. After that, the cells were fixed and the VP1 protein was detected by immunofluorescence (FIG. 5). The VP1 was detected by means of fluorescence-microscopic methods using a VP1-specific immune serum and subsequently incubating with an FITC-coupled anti-rabbit Mab. In all the four cell lines, it was possible to detect marked binding of VP1-VLP/PEI-transferrin to the cell membrane following the incubation. In the HeLa cells, it was possible to detect a marked concentration of VP1 at the nuclear membrane only 2 hours after incubating with VP1-VLP/PEI-transferrin. It was not possible to observe any binding of VP1-VLP to the cell membrane, in any of the cell lines investigated, in control experiments, which were carried out without any previous treatment with PEI-transferrin.

### Example 4

### Transducing Various Cells Using VP1-VLP/PEI-Transferrin

The transduction efficiency of VP1-VLP/PEI-transferrin was determined by detecting expression of the reporter gene luciferase. For this, 1 μg of the plasmid pGL3-C (Promega) was packaged in 1.25 μg of VP1-VLP. The VP1-VLPs were subsequently loaded with 1.9 μg of PEI-transferrin, as described in Example 2. The VP1-VLP/PEI-transferrin complexes were incubated with the cell lines HeLa, DU145, BM 1604 and BPH-1, as described in Example 3. In each case 5×10⁴ cells were incubated with the VLP complexes for 24 hours and then washed. After that, the medium was changed and the cells were cultured for a further 24 hours. Expression of the luciferase was detected quantitatively in the cell lysates by means of luminometry using a commercially available system (Promega). As controls, unloaded VLPs, and also DNA/PEI-transferrin, were also used, in addition to the VLP complexes, for transducing the cells.

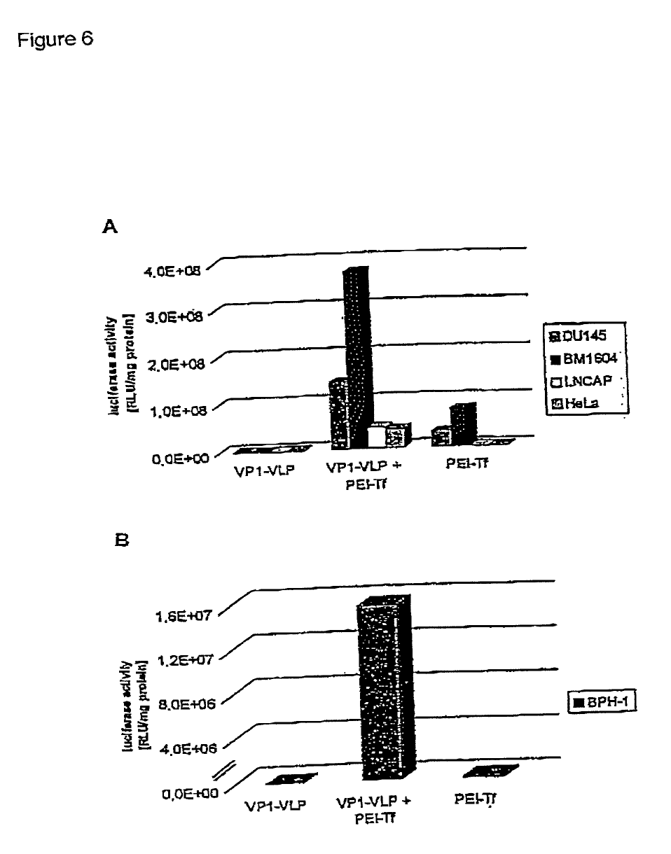

The results of the luciferase measurements provide an impressive demonstration that, while the cells cannot be transduced using unmodified VP1-VLPs, they are efficiently transduced when the VLPs have been previously loaded with PEI-transferrin (FIG. 6). In addition to this, the luciferase activity data show that transduction using VP1-VLP/PEI-transferrin complexes is more efficient than using conventional DNA bound to PEI-transferrin.

These investigations demonstrated unambiguously that cationic polymers may be used for anchoring ligands on the VP1-VLPs, that cell-specific ligands can be used to alter the narrow target cell tropism of the VP1-VLPs, and that the efficiency of transduction and expression is markedly increased as compared with conventionally employed PEI.

The disclosure in German Patent Application 101 31 145.1 of Jun. 28, 2001 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in compositions for transferring active compounds in a cell-specific manner, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A virus-like particle (VLP) comprising a plurality of molecules of viral protein (VP1) from JC virus and a cationic polymer associated with said molecules of said viral protein.

2. The virus-like particle as defined in claim 1, comprising recombinant viral protein (VP1).

3. The virus-like particle as defined in claim 1, wherein the viral protein (VP1) is encoded by a nucleic acid and said nucleic acid has nucleotide sequence as shown in SEQ. ID NO. 1, or a nucleotide sequence complementary thereto, or another nucleotide sequence corresponding to said nucleotide sequence SEQ. ID NO. 1 or said nucleotide sequence complementary thereto, within the context of genetic code degeneracy, or a further nucleotide sequence which hybridizes with one of the aforesaid nucleotide sequences under stringent conditions.

4. The virus-like particle as defined in claim 1, wherein said cationic polymer is a polyamine or polyimine.

5. The virus-like particle as defined in claim 1, wherein said cationic polymer comprises an amino acid polymer.

6. The virus-like particle as defined in claim 1, wherein said cationic polymer is polylysine.

7. The virus-like particle as defined in claim 1, wherein said cationic polymer is a polyalkylenimine.

8. The virus-like particle as defined in claim 1, wherein said cationic polymer is a polyethylenimine (PEI).

9. The virus-like particle as defined in claim 1, further comprising at least one ligand bound to the cationic polymer.

10. The virus-like particle as defined in claim 1, further comprising at least one ligand bound to the cationic polymer and wherein said at least one ligand comprises a target cell-specific group.

11. The virus-like particle as defined in claim 1, further comprising at least one ligand bound to the cationic polymer and wherein said at least one ligand comprises a target cell-specific group and said target cell-specific group consists of a binding partner for a cell surface receptor.

12. The virus-like particle as defined in claim 1, further comprising at least one ligand bound to the cationic polymer and wherein said at least one ligand comprises a labeling group.

13. The virus-like particle as defined in claim 1, further comprising at least one ligand bound to the cationic polymer and wherein said at least one ligand comprises an effector group.

14. The virus-like particle as defined in claim 1, having a capsid structure and further comprising at least one active substance within the capsid structure.

15. The virus-like particle as defined in claim 1, having a capsid structure and further comprising at least one active substance within the capsid structure and wherein the at least one active substance is selected from the group consisting of nucleic acids, proteins and physiologically active substances.

16. The virus-like particle as defined in claim 1, having a capsid structure and further comprising at least one active substance within the capsid structure and wherein the at least one active substance has been packaged therein by means of a dissociation/re-association cycle.

17. A process for preparing a virus-like particle (VLP) comprising a plurality of molecules of viral protein (VP1) from JC virus and a cationic polymer associated with said molecules of said viral protein, said process comprising the steps of:
a) assembling said plurality of said molecules of said viral protein (VP1), and
b) at least one of before, during and after the assembling of step a), adding said cationic polymer for association with said molecules in order to form said virus-like particle (VLP).

18. The process as defined in claim 17, wherein the adding takes place after the assembling.

19. The process as defined in claim 18, wherein the assembling is carried out in the presence of an additional substance so that the additional substance is enclosed within a capsid coat of said virus-like particle.

20. The process as defined in claim 17, further comprising encoding the viral protein (VP1) with a nucleic acid and wherein said nucleic acid has nucleotide sequence as shown in SEQ. ID NO. 1, or a nucleotide sequence complementary thereto, or another nucleotide sequence corresponding to said nucleotide sequence shown in said SEQ. ID NO. 1 or said nucleotide sequence complementary thereto, within the context of genetic code degeneracy, or a further nucleotide sequence which hybridizes with one of the aforesaid nucleotide sequences under stringent conditions.

21. A diagnostic or therapeutic agent consisting of at least one virus-like particle (VLP) and wherein said at least one virus-like particle comprises a plurality of molecules of viral protein (VP1) from JC virus and a cationic polymer associated with said molecules of said viral protein.

22. The diagnostic or therapeutic agent as defined in claim 21, wherein the viral protein (VP1) is encoded by a nucleic acid and said nucleic acid has nucleotide sequence as shown in SEQ. ID NO. 1, or a nucleotide sequence complementary thereto, or another nucleotide sequence corresponding to said nucleotide sequence shown in SEQ. ID NO. 1 or said nucleotide sequence complementary thereto, within the context of genetic code degeneracy, or a further nucleotide sequence which hybridizes with one of the aforesaid nucleotide sequences under stringent conditions.

23. A transport vehicle for transporting at least one active compound into a target cell, said transport vehicle consisting of at least one virus-like particle (VLP) and wherein said at least one virus-like particle comprises a plurality of molecules of viral protein (VP1) from JC virus and a cationic polymer associated with said molecules of said viral protein.

24. The transport vehicle as defined in claim 23, wherein the viral protein (VP1) is encoded by a nucleic acid and said nucleic acid has nucleotide sequence as shown in SEQ. ID NO. 1, or a nucleotide sequence complementary thereto, or another nucleotide sequence corresponding to said nucleotide sequence shown in SEQ. ID NO. 1 or said nucleotide sequence complementary thereto, within the context of genetic code degeneracy, or a further nucleotide sequence which hybridizes with one of the aforesaid nucleotide sequences under stringent conditions.

25. The transport vehicle as defined in claim 23, wherein the at least one active compound is a nucleic acid.

26. The transport vehicle as defined in claim 23, comprising means for specifically transducing cells.

27. The transport vehicle as defined in claim 25, comprising means for gene therapy.

28. A method of specifically transducing cells of renal or neuronal origin, said method comprising using a plurality of virus-like particles and wherein said virus-like particles are each composed of a plurality of molecules of viral protein (VP1) from JC virus.

29. A method of specifically transporting an active compound into cells of renal or neuronal origin, said method comprising using a plurality of virus-like particles and wherein said virus-like particles are each composed of a plurality of molecules of viral protein (VP1) from JC virus.

30. The method as defined in claim 29, wherein said active compound is a nucleic acid.

31. The method as defined in claim 30, further comprising a method for gene therapy.
